SEQUENCE LISTING (4 sequences)

SEQ ID NO: 1 - DNA, 15 bp, Artificial Sequence
  Oligonucleotide
aacgttgaggggcat

SEQ ID NO: 2 - DNA, 15 bp, Artificial Sequence
  Oligonucleotide
gaacggagacggttt

SEQ ID NO: 3 - DNA, 20 bp, Artificial Sequence
  Oligonucleotide
tcccgcctgtgacatgcatt

SEQ ID NO: 4 - DNA, 20 bp, Artificial Sequence
  Oligonucleotide
tcccgcgcacttgatgcatt

(19) United States
(12) Patent Application Publication
(10) Pub. No.: US 2006/0147514 A1
(43) Pub. Date: Jul. 6, 2006

(54) Method for introducing antisense oligonucleotides into eucaryotic cells

(75) Inventors: Gulilat Gebeyehu, Potomac, MD (US); Donna K. Fox, Sykesville, MD (US); Martha K. Ogilvie, Washington, DC (US)

Correspondence Address: GREENLEE WINNER AND SULLIVAN P C, 4875 PEARL EAST CIRCLE, SUITE 200, BOULDER, CO, 80301, US

(73) Assignee: Invitrogen Corporation, Carlsbad, CA (US)

(21) Appl. No.: 11/369,715

(22) Filed: Mar. 6, 2006

Related U.S. Application Data
(62) Division of application No. 09/984,076, filed on Oct. 26, 2001.
(60) Provisional application No. 60/243,069, filed on Oct. 27, 2000.

(51) Int. Cl. A61K 48/00; A61K 9/127; C12N 15/88

(52) U.S. Cl. 424/450; 514/44; 435/458

(57) ABSTRACT

The present invention relates to a method for introducing one or more antisense oligonucleotides into one or more eucaryotic cells using one or more lipid formulations comprising one or more cationic lipids of Formula I and optionally at least one neutral lipid. In particular, the present invention relates to a method for introducing one or more antisense oligonucleotides into one or more eucaryotic cells using a lipid formulation comprising dimethyldioctadecylammonium bromide (DDAB) and at least one neutral lipid, especially dioleylphosphatidylethanolamine (DOPE). The invention also relates to kits for carrying out the invention, compositions for carrying out the invention, and compositions formed while carrying out the invention. Further, the present invention relates to a method for inhibiting or preventing cell growth or proliferation, and a method for inhibiting or preventing expression of one or more proteins.

### CROSS-REFERENCE TO RELATED APPLICATIONS

This application claims the benefit of the filing date of U.S. Provisional Application No. 60/243,069, filed Oct. 27, 2000, the entirety of which is incorporated by reference herein.

### BACKGROUND OF THE INVENTION

1. Field of the Invention

The present invention relates to a method for introducing one or more antisense oligonucleotides into one or more eucaryotic cells using one or more lipid formulations comprising one or more cationic lipids of Formula I and optionally at least one neutral lipid. In particular, the present invention relates to a method for introducing one or more antisense oligonucleotides into one or more eucaryotic cells using a lipid formulation comprising dimethyldioctadecylammonium bromide (DDAB) and at least one neutral lipid, especially dioleylphosphatidylethanolamine (DOPE). The invention also relates to kits for carrying out the invention, compositions for carrying out the invention, and compositions formed while carrying out the invention. Further, the present invention relates to a method for inhibiting or preventing cell growth or proliferation, and a method for inhibiting or preventing expression of one or more proteins.

2. Related Art

Antisense oligonucleotides have been described in the art as naturally occurring biological inhibitors of gene expression in both prokaryotes (Mizuno et al., Proc. Natl Acad Sci. USA 81:1966-1970 (1984)) and eukaryotes (Heywood, Nucleic Acids Res. 14:6771-6772 (1986)), and these sequences presumably function by hybridizing to complementary mRNA sequences, resulting in hybridization arrest of translation (Paterson, et al.,

Proc. Natl. Acad. Sci. USA, 74:4370-4374 (1987)).

Antisense oligonucleotides are short synthetic DNA or RNA nucleotide molecules formulated to be complementary to a specific gene or RNA message. Through the binding of these oligomers to a target DNA or mRNA sequence, transcription or translation of the gene can be selectively blocked and the disease process generated by that gene can be halted (see, for example, Jack Cohen, Oligodeoxynucleotides, Antisense Inhibitors of Gene Expression, CRC Press (1989)). The cytoplasmic location of mRNA provides a target considered to be readily accessible to antisense oligodeoxynucleotides entering the cell; hence much of the work in the field has focused on RNA as a target. Currently, the use of antisense oligodeoxynucleotides provides a useful tool for exploring regulation of gene expression in vitro and in tissue culture (Rothenberg et al., J. Natl. Cancer Inst. 81:1539-1544 (1989)).

Antisense therapy is the administration of exogenous oligonucleotides which bind to a target polynucleotide located within the cells. For example, antisense oligonucleotides may be administered systemically for anticancer therapy (WO 90/09180). Antisense oligonucleotides are administered to a patient in order to inhibit the expression of the corresponding protein.

U.S. Pat. No. 5,279,833 describes a reagent for introducing nucleic acids into an animal cell. The reagent comprises a neutral lipid, such as dioleyl phosphatidylethanolamine (DOPE), and a cationic lipid, such as an ammonium salt of formula

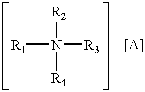

wherein R₁ is a straight hydrocarbon chain of C₁₄ to C₁₈ that is saturated or unsaturated, R₂, R₃ and R₄ are, independently of each other, hydrogen, a straight hydrocarbon chain of C₁-C₁₈ that is saturated or unsaturated or an aryl, e.g., benzyl or phenyl, an A is an anion. The patent describes cetyldimethylethylammonium bromide and dimethyldioctadecylammonium bromide (DDAB) as preferred ammonium salts.

Liu et al, J. Biol. Chem. 272:11690-11693 (1997) describe an antisense oligonucleotide treatment of drug-resistant human breast carcinoma (MCF-7/ADR) cells, wherein the antisense mixture was made by combining solution A containing 20 mg/ml LIPOFECTACE™ in 0.25 ml of McCoy's 5A medium without serum and solution B containing 400 nM of the antisense oligonucleotide in 0.25 ml of McCoy's 5A medium without serum. LIPOFECTACE contains DDAB and DOPE in the ratio of 1:2.5. However, the disclosed concentration of LIPOFECTACE™ reagent (20 mg/ml) is impossible to achieve because of solubility problems. Further, Liu et al. state that the transfections were performed according to the manufacturer's instructions. Contrary to this, LIPOFECTACE™ does not include instructions for antisense oligonucleotide transfection.

U.S. Pat. No. 5,753,613 describes compositions for introducing a polyanionic material into a cell comprising a cationic compound of formula I 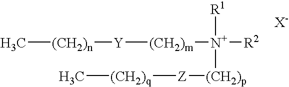
wherein R¹ and R² are independently C₁₋₁₃ alkyl and Y and Z are independently members selected from the group consisting of —CH₂CH₂CH₂CH₂CH₂—, —CH═CHCH₂CH₂CH₂—, —CH₂CH═CHCH₂CH₂—, —CH₂CH₂CH═CHCH₂—, —CH₂CH₂CH₂CH═CH—, —CH═CHCH═CHCH₂—, —CH═CH₂CH₂CH═CH—, and —CH₂CH═CHCH═CH—, with the proviso that Y and Z are not both —CH₂CH₂CH₂CH₂CH₂—; n and q are independently integers of from 3 to 7; and m and p are independently integers of from 4 to 9, with the proviso that the sums n+m and q+p are each integers of from 10 to 14 and X is an anion. U.S. Pat. No. 5,753,613 describes that these compositions can be used, e.g., for introducing antisense oligonucleotides in the cells. It is further described that DDAB has a poor transfection efficiency.

There is great potential for the use of antisense oligonucleotides to regulate gene expression. However, factors that often limit the efficacy of antisense oligonucleotides include inefficient cellular uptake, toxicity of the delivery agent, and non-specific effects seen with control oligonucleotides (Neckers, L. M., Antisense Research and Applications, CRC Press (1993) 451 and Giles, R. V., Current Opinions in Molecular Therapeutics 2:238-252 (2000)). Thus, a need exists in the art for an efficient and non-toxic method for introducing antisense oligonucleotides into eucaryotic cells.

### SUMMARY OF THE INVENTION

Applicants have discovered that lipid formulations comprising one or more cationic lipids of Formula I (below) are ideal for introducing one or more antisense oligonucleotides into eucaryotic cells. Applicants have found that when a lipid formulation comprising one or more cationic lipids of Formula I and optionally at least one neutral lipid is contacted with an antisense oligonucleotide, a stable complex is formed with the antisense oligonucleotide which permits efficient delivery of the antisense oligonucleotide into an eucaryotic cell. Further, introducing antisense oligonucleotides into eucaryotic cells using the above formulations can be accomplished without inducing cytotoxicity which is a serious problem in the field of antisense technology. Accordingly, the invention provides a method for introducing one or more antisense oligonucleotides into one or more eucaryotic cells, comprising

(a) contacting said one or more antisense oligonucleotides with one or more lipid formulations comprising an effective amount of one or more cationic lipids of Formula I 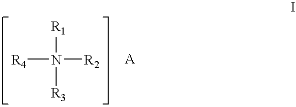
wherein

R₁ is a straight or a branched hydrocarbon chain of C₁₀₋₁₀₀ that is saturated or unsaturated;

R₂ is selected from the group consisting of a pair of electrons, hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted;

R₃ and R₄, independently of one another, are selected from the group consisting of hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted; wherein R₅ and R₆ are independently alkylene, alkenylene or alkynylene; and

A is a pharmaceutically acceptable anion when R₂ is not a pair of electrons;

and optionally at least one neutral lipid to form one or more antisense oligonucleotide-lipid aggregate complexes, and

(b) contacting said one or more cells with said one or more complexes.

In a preferred aspect, R₁ is a straight or a branched hydrocarbon chain of C₁₀₋₃₀ that is saturated or unsaturated. In another preferred aspect, when R₃ and R₄ in Formula I are C₁₋₃ alkyl, and one of R₁ or R₂ is an unsaturated C₁₆₋₂₀ alkyl, the other one of R₁ and R₂ is not an unsaturated or saturated C₁₆₋₂₀ alkyl.

In a further preferred aspect, the one or more eucaryotic cells are not drug-resistant human breast carcinoma cells.

Also, the invention provides a method for introducing one or more antisense oligonucleotides into one or more eucaryotic cells, comprising

(a) contacting said one or more antisense oligonucleotides with one or more lipid formulations comprising an effective amount of one or more cationic lipids of Formula II 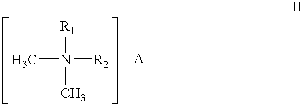
wherein

R₁ is a straight or a branched hydrocarbon chain of C₁₀₋₁₀₀ that is saturated or unsaturated;

R₂ is selected from the group consisting of a pair of electrons, hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted, wherein R₅ and R₆ are independently alkylene, alkenylene or alkynylene; and

A is a pharmaceutically acceptable anion when R₂ is not a pair of electrons;

and optionally at least one neutral lipid to form one or more antisense oligonucleotide-lipid aggregate complexes, and

(b) contacting said one or more cells with said one or more complexes.

In a preferred aspect, R₁ is a straight or a branched hydrocarbon chain of C₁₀₋₃₀ that is saturated or unsaturated. In another preferred aspect, when one of R₁ or R₂ in Formula II is an unsaturated C₁₆₋₂₀ alkyl, the other one is not an unsaturated or saturated C₁₆₋₂₀ alkyl.

In particular, the invention provides a method for introducing one or more antisense oligonucleotides into one or more eucaryotic cells, comprising

(a) contacting said one or more antisense oligonucleotides with a lipid formulation comprising an effective amount of dimethyldioctadecylammonium bromide (DDAB) and at least one neutral lipid to form one or more antisense oligonucleotide-lipid aggregate complexes , and

(b) contacting said one or more cells with said one or more complexes.

The invention also concerns a kit, wherein the kit is preferably used for introducing one or more oligonucleotides into one or more eucaryotic cells, such kit preferably comprising at least one component selected from the group consisting of one or more cells, one or more antisense oligonucleotides, one or more lipid formulations of the invention, one or more buffering salts, one more culture media, and one or more transfection enhancers.

The invention also relates to a composition for carrying out the method of the present invention, and the composition formed while carrying out the invention. Such compositions may comprise at least one component selected from the group consisting of one or more cells, one or more antisense oligonucleotides, one or more lipid formulations of the invention, one or more buffering salts, one more culture media, and one or more transfection enhancers.

Further, the invention provides a method for inhibiting or preventing cell growth or proliferation, comprising

(a) contacting one or more eucaryotic cells with one or more antisense oligonucleotides and an effective amount of one or more lipid formulations comprising an effective amount of one or more cationic lipids of Formula I and optionally at least one neutral lipid to provide a composition; and

(b) incubating said composition under conditions sufficient to inhibit or prevent cell growth or proliferation.

Furthermore, the invention provides a method for inhibiting or preventing expression of one or more proteins, comprising

(a) contacting one or more eucaryotic cells with one or more antisense oligonucleotides and an effective amount of one or more lipid formulations comprising an effective amount of one or more cationic lipids of Formula I and optionally at least one neutral lipid to provide a composition; and

(b) incubating said composition under conditions sufficient to inhibit or prevent said expression of one or more proteins.

Additional embodiments and advantages of the invention will be set forth in part in the description as follows, and in part will be obvious from the description, or may be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and not restrictive of the invention, as claimed.

### BRIEF DESCRIPTION OF THE FIGURES

FIG. 1 is a graph showing the inhibition of proliferation TR0/anti-c-myc complexes in different cell lines. The black column represents the untreated sample. The white column represents cells that received only lipid and no oligonucleotide. The hatched column represents cells that received the scrambled control. The gray column represents cells that received antisense oligonucleotide.

FIG. 2 compares the ability of various transfection reagents to mediate functional oligonucleotide transfection. The black column represents untreated sample. The white column represents cells that received only lipid and no oligonucleotide. The hatched column represents cells that received the scrambled control. The gray column represents cells that received antisense oligonucleotide.

FIG. 3 depicts an immunoblot analysis of c-Raf protein expression in HeLa cells treated with antisense (AS) or mismatched (MM) oligonucleotides in comparison to untreated controls. Lane 1 is a cell extract from untreated HeLa cells. Lane 2 is a cell extract that received TR0 but no ODN. Lane 3 is a cell extract that received the TR0/antisense ODN to c-raf complex and Lane 4 is the TR0/mismatch control ODN complex.

### DETAILED DESCRIPTION OF THE PREFERRED EMBODIMENTS

Applicants have surprisingly discovered an efficient and non-toxic method for introducing antisense oligonucleotides into eucaryotic cells. Accordingly, the invention relates to a method for introducing one or more antisense oligonucleotides into one or more eucaryotic cells, comprising

(a) contacting said one or more antisense oligonucleotides with one or more lipid formulations comprising one or more cationic lipids of Formula I 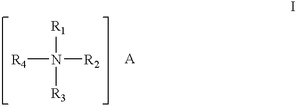
wherein

R₁ is a straight or a branched hydrocarbon chain of C_{10 100} that is saturated or unsaturated;

R₂ is selected from the group consisting of a pair of electrons, hydrogen, alkyl, alkenyl, alkynyl, heteroalkyl, heteroalkenyl, heteroalkynyl, arylalkyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkenyl, arylalkynyl, and aryl, all of which can be optionally substituted;

R₃ and R₄, independently of one another, are selected from the group consisting of hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which may be optionally substituted, wherein R₅ and R₆ are independently alkylene, alkenylene or alkynylene; and

A is a pharmaceutically acceptable anion when R₂ is not a pair of electrons;

and optionally at least one neutral lipid to form one or more antisense oligonucleotide-lipid aggregate complexes, and

(b) contacting said one or more cells with said one or more complexes.

Preferably, when R₃ and R₄ in Formula I are C₁₋₃ alkyl, and one of R₁ or R₂ is an unsaturated C₁₆₋₂₀ alkyl, the other one of R₁ and R₂ is not an unsaturated or saturated C₁₆₋₂₀ alkyl. Preferably, the one or more cells are not drug-resistant human breast carcinoma cells. Preferably 1-5 antisense oligonucleotides, more preferably 1-3 antisense oligonucleotides, especially one antisense oligonucleotide, are contacted with one or more lipid formulations.

Preferably, R₁ is a straight or a branched hydrocarbon chain of C₁₀₋₃₀ that is saturated or unsaturated. Preferably, R₁ is a straight hydrocarbon chain of C₁₂₋₂₄ that is saturated or unsaturated; and R₂, R₃ and R₄ are independently selected from the group consisting of hydrogen, C₁₋₂₀ alkyl, C₂₋₂₀ alkenyl, C₂₋₂₀ alkynyl, C₄₋₂₀ heteroalkyl, C₄₋₂₀ heteroalkenyl, C₄₋₂₀ heteroalkynyl, C₆₋₁₂ aryl(C₁₋₂₀) alkyl and C₆₋₁₂ aryl, all of which can be optionally substituted. More preferably, R₁ is a straight hydrocarbon chain of C₁₄₋₂₀ that is saturated or unsaturated; R₂ is selected from the group consisting of hydrogen, C₆₋₁₈ alkyl, C₆₋₁₈ alkenyl, C₆₋₁₈ alkynyl, C₆₋₁₈ heteroalkyl, C₆₋₁₈ heteroalkenyl, C₆₋₁₈ heteroalkynyl, phenyl(C₆₋₁₈)alkyl, and phenyl; and R₃ and R₄ are independently selected from the group consisting of hydrogen, C₁₋₅ alkyl, C₂₋₆ alkenyl, C₂₋₆ alkynyl, C₂₋₅ heteroalkyl, C₂₋₅ heteroalkenyl, C₂₋₅ heteroalkynyl, phenyl(C₁₋₅)alkyl, especially benzyl, and phenyl, all of which can be optionally substituted.

A useful group of cationic lipids of Formula I include those wherein R₁ and R₂ are both C₁₀₋₂₀ saturated alkyl groups.

Useful cationic lipids in the present invention included in Formula I are cationic lipids of Formula II 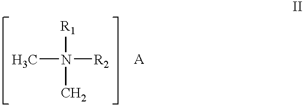
wherein

R₁ is a straight or a branched hydrocarbon chain of C₁₀₋₁₀₀ that is saturated or unsaturated;

R₂ is selected from the group consisting of a pair of electrons, hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted, wherein R₅ and R₆ are independently alkylene, alkenylene or alkynylene; and

A is a pharmaceutically acceptable anion when R₂ is not a pair of electrons.

Preferably, when one of R₁ or R₂ in Formula II is an unsaturated C₁₆₋₂₀ alkyl, the other one is not an unsaturated or saturated C₁₆₋₂₀ alkyl.

Preferably, R₁ in Formula II is a straight or a branched hydrocarbon chain of C₁₀₋₃₀ that is saturated or unsaturated. Preferably, R₁ in Formula II is a straight hydrocarbon chain of C₁₂₋₂₄ that is saturated or unsaturated; and R₂ is selected from the group consisting of hydrogen, C₁₋₂₀ alkyl, C₂₋₂₀ alkenyl, C₂₋₂₀ alkynyl, C₄₋₂₀ heteroalkyl, C₄₋₂₀ heteroalkenyl, C₄₋₂₀ heteroalkynyl, C₆₋₁₂ aryl(C₁₋₂₀) alkyl and C₆₋₁₂ aryl, all of which can be optionally substituted. More preferably, R₁ is a straight hydrocarbon chain of C₁₄₋₂₀ that is saturated, and R₂ is selected from the group consisting of C₆₋₁₈ alkyl, C₆₋₁₈ heteroalkyl, C₆₋₁₈ heteroalkenyl, C₆₋₁₈ heteroalkynyl, and phenyl(C₆₋₁₈)alkyl, all of which can be optionally substituted.

A is any pharmaceutically acceptable anion. These anions can be organic or inorganic. A is preferably a halogen, that is Br⁻, Cl³¹ , F⁻, I⁻, or A is a sulfate, a nitrite or a nitrite.

Preferably the cationic lipid of Formula I is dimethyldioctadecylammonium bromide (DDAB).

Preferably, the lipid formulation contains at least one neutral lipid. Examples of neutral lipids which can be used in the present formulations are, for example, diacylphosphatidylcholine, diacylphosphatidylethanolamine, ceramide, sphingomyelin, phosphatidic acid, and cholesterol. Preferably, the present formulations contain at least one neutral lipid selected from the group consisting of diacylphosphatidylcholine, such as dioleyphosphatidylcholine, dipalmitoylphosphatidylcholine, palmitoyloleylphosphatidylcholine, lecithin and lysolecithin, diacylphosphatidylethanolamine, ceramide, sphingomyelin, and cholesterol. More preferably, the neutral lipid is a diacylphosphatidylethanolamine having 10-24 carbon atoms in the acyl group. More preferably the acyl groups are lauroyl, myristoyl, heptadecanoyl, palmitoyl, stearoyl or oleyl. Especially, the neutral lipid is dioleylphosphatidylethanolamine (DOPE), palmitoyloleylphosphatidylethanolamine, diheptadecanoylphosphatidylethanolamine,, dilauroylphosphatidylethanolamine, dimyristoylphosphatidylethanolamine, distearoylphosphatidylethanolamine, beta-linoleyl-gamma-palmitoylphosphatidylethanolamine, and beta-oleyl-gamma-palmitoylphosphatidylethanolamine, specifically dioleylphosphatidylethanolamine (DOPE).

The ratio of the cationic lipid of Formula I or II to a neutral lipid can be widely varied depending on the particular cationic lipid employed. For example, the ratio can be from about 1:10 to about 1: 1, preferably from about 1:5 to about 1:2.5.

The ratio of antisense oligonucleotides to cationic lipids of Formula I or II should not be so high as to saturate the positive charges on the lipid aggregates, which may result in a lack of binding of the lipid aggregates to the cell surface.

The lipid formulation containing one or more cationic lipids of Formula I and optionally at least one neutral lipid can be present in an amount of about 0.1 μg/ml-5 mg/ml when the antisense oligonucleotide is contacted with the lipid formulation. Preferably, the lipid formulation is present in an amount of 0.15 μg/ml-4.5 mg/ml, more preferably 0.15 μg/ml-4.2 mg/ml, more preferably 0.15 μg/ml-4.0 mg/ml, more preferably 0.2 μg/ml-3.7 mg/ml, more preferably 0.2 μg/ml-3.5 mg/ml, more preferably 0.2 μg/ml-3.2 mg/ml, more preferably 0.25 μg/ml-3.0 mg/ml, more preferably 0.25 μg/ml-2.8 mg/ml, more preferably 0.25 μg/ml-2.5 mg/ml, more preferably 0.25 μg/ml-2.3 mg/ml, more preferably 0.3 μg/ml-2.0 mg/ml, more preferably 0.3 μg/ml-1.8 mg/ml, more preferably 0.3 μg/ml-1.6 mg/ml, more preferably 0.3 μg/ml-1.4 mg/ml, 0.3 μg/ml-1.1 mg/ml, more preferably 0.35 μg/ml-0.8 mg/ml, more preferably 0.35 μg/ml-0.5 mg/ml, 0.35 μg/ml-0.3 mg/ml, more preferably 0.35 μg/ml-0.1 mg/ml, more preferably 0.35-90 μg/ml, more preferably 0.35-75 μg/ml, more preferably 0.35-60 μg/ml, more preferably 0.35-45 μg/ml, more preferably 0.35-30 μg/ml, more preferably 0.35-20 μg/ml, more preferably 0.35-14 μg/ml, more preferably 0.7-14 μg/ml, more preferably about 1-14 μg/ml, more preferably about 2-13 μg/ml, more preferably about 3-13 μg/ml, more preferably about 4-12 μg/ml, especially about 4.5-12 μg/ml.

In a preferred embodiment, the invention relates to a method for introducing one or more antisense oligonucleotides into one or more eucaryotic cells, comprising

(a) contacting said one or more antisense oligonucleotides with a lipid formulation comprising an effective amount of dimethyldioctadecylammonium bromide (DDAB) and at least one neutral lipid to form one or more antisense oligonucleotide-lipid aggregate complexes, and

(b) contacting said one or more cells with said one or more complexes.

Preferably, the neutral lipid is diacylphosphatidylethanolamine having 10-24 carbon atoms in the acyl group, more preferably dioleylphosphatidylethanolamine (DOPE). Preferably, the ratio of DDAB:DOPE in the present method is from about 1:5 to about 1:1, more preferably 1:2.5. Preferably, the final concentration of the lipid formulation comprising DDAB and DOPE in the ratio of 1:2.5 is 5.6-11.2 μg/ml.

The present invention also relates to a kit, wherein the kit is preferably used for introducing one or more oligonucleotides into one or more eucaryotic cells. Such kit preferably comprises at least one component selected from the group consisting of one or more cells, one or more antisense oligonucleotides, one or more lipid formulations of the invention, one or more buffering salts, one more culture media, and one or more transfection enhancers. More preferably, such kit comprises one or more lipid formulations comprising an effective amount of one or more cationic lipids of Formula I and optionally at least one neutral lipid, and at least one additional component selected from the group consisting of one or more cells, one or more antisense oligonucleotides, one or more buffering salts, one or more culture media, and one or more transfection enhancers. Such kit may further include one or more cell-targeting enhancers, uptake enhancers, internalization enhancers, nuclear targeting enhancers and expression enhancers.

The invention also relates to a composition for carrying out the method of the present invention, and the composition formed while carrying out the invention. Such compositions may comprise at least one component selected from the group consisting of one or more cells, one or more antisense oligonucleotides, one or more lipid formulations of the invention, one or more buffering salts, one more culture media, and one or more transfection enhancers. Preferably, such compositions comprise one or more lipid formulations comprising an effective amount of one or more cationic lipids of Formula I and optionally at least one neutral lipid, and one or more additional components selected from the group consisting of one or more cells, one or more antisense oligonucleotides, one or more buffering salts, one or more culture media, and one or more transfection enhancers. Such compositions may further include one or more cell-targeting enhancers, uptake enhancers, internalization enhancers, nuclear targeting enhancers and expression enhancers.

Further, the invention relates to a method for inhibiting or preventing cell growth or proliferation, comprising

(a) contacting one or more eucaryotic cells with one or more antisense oligonucleotides and an effective amount of one or more lipid formulations comprising an effective amount of one or more cationic lipids of Formula I and optionally at least one neutral lipid to provide a composition; and

(b) incubating said composition under conditions sufficient to inhibit or prevent cell growth or proliferation.

Furthermore, the invention relates to a method for inhibiting or preventing expression of one or more proteins, comprising

(a) contacting one or more eucaryotic cells with one or more antisense oligonucleotides and an effective amount of one or more lipid formulations comprising an effective amount of one or more cationic lipids of Formula I and optionally at least one neutral lipid to provide a composition; and

(b) incubating said composition under conditions sufficient to inhibit or prevent said expression of one or more proteins.

Some compounds of Formula I, such as DDAB, are commercially available. Compounds of Formula I can be prepared by methods known to those of skill in the art using standard synthetic reactions (see March, Advanced Organic Chemistry, 4^{th} Ed., Wiley-Interscience, New York, N.Y. (1992)). For example, compounds of Formula I, wherein R₁-R₄ are the same or different, can be prepared treating a C₁₀₋₁₀₀ amine, preferably a C₁₀₋₃₀ amine, with formaldehyde and sodium cyanoborohydride under conditions that result in the reductive alkylation of the amine to provide a tertiary amine which further is reacted with, e.g., an optionally substituted alkyl bromide to provide a quaternary ammonium salt. Further, compounds of Formula I can be prepared by converting a fatty acid to its corresponding acid chloride with, e.g., oxalyl chloride, thionyl chloride, p-TsCl, PCl₃ or PCl₅, and reacting the acid chloride with an optionally substituted amine to provide a corresponding amide. Reduction of the amide with, e.g., lithium aluminium hydride provides a secondary amine. The secondary amine is further treated with optionally substituted alkyl halides to provide the quaternary ammonium salt. Anion exchange can then be carried to out to provide cationic lipids having the desired pharmaceutically acceptable anion.

Certain of the cationic lipids of Formula I may be insufficiently soluble in physiological media to employ for the method of the present invention. Those of ordinary skill in the art will appreciate that there are a variety of techniques available in the art to enhance solubility of such compounds in aqueous media, such as using ethanol as a co-solvent. Such methods are readily applicable without undue experimentation to the compounds described herein.

In the method of the present invention, one or more cationic lipids of Formula I are used in combination with optionally at least one neutral lipid to prepare liposomes, micelles and other lipid aggregates suitable for introducing antisense oligonucleotides into target cells, either in vitro or in vivo. Such lipid aggregates are polycationic, and are able to form stable complexes with antisense oligonucleotides. The lipid aggregate oligonucleotide complex interacts with cells making the antisense oligonucleotide available for absorption and uptake by the cell.

Liposomes and micelles containing one or more cationic lipids of Formula I and optionally at least one neutral lipid can be prepared by methods well known in the art. The selection of neutral lipids is generally guided by consideration of, e.g., liposome size and stability of the liposomes in the bloodstream. Liposomes can be generally formed by sonicating a lipid in an aqueous medium, by resuspension of dried lipid layers in a buffer or by dialysis of lipids dissolved in an organic solvent against a buffer of choice. Another method of liposome preparation is utilizing microfluidization. In this process, one or more cationic lipids of Formula I and optionally at least one neutral lipid are mixed in an organic solvent, such as chloroform. The organic solvent is removed by evaporation to leave a lipid film. The lipid film is hydrated with water and past through a microfluidizer. By selecting the appropriate ratio, various sizes of liposomes can be prepared. For example, liposomes can be prepared as described in Szoka et al., Ann. Rev. Biophys. Bioeng. 9:467 (1980), U.S. Pat. Nos. 4,235,871, 4,501,728, and 4,837,028, the text Liposomes, Marc J. Ostro, ed., Marcel Dekker, Inc., New York, 1983, Chapter 1, and Hope et al., Chem. Phys. Lip. 40:89 (1986).

Following liposome preparation, the liposomes may be sized to achieve a desired range and relatively narrow distribution of liposome sizes. Several techniques are available for sizing liposomes to a desired size. One sizing method is described, for example, in U.S. Pat. No. 4,737,323. Liposomes typically range in diameter from 250 angstrom units to several micrometers (the diameter of a red blood cell is roughly 10 micrometers) and are usually suspended in solution. They have two standard forms: “onion-skimmed” multilamellar vesicles (MLV's), made up of several lipid bilayers separated by fluid, and unilamellar vesicles, consisting of single bilayer surrounding an entirely fluid core. The unilamellar vesicles are typically characterized as being small (SUV's) or large (LUV's).

Under appropriate circumstances liposomes can absorb to almost any cell type. Once they have been adsorbed, liposomes may be endocytosed, or swallowed up, by some cells. Adsorbed liposomes can also exchange lipids with cell membranes and may at times be able to fuse with cells. When fusion takes place, the liposomal membrane is integrated into the cell membrane and the aqueous contents of the liposome merge with the fluid in the cell.

Endocytosis of liposomes occurs in a limited class of cells; those that are phagocytic, or able to ingest foreign particles. When phagocytic cells take up liposomes, the cells move the spheres into subcellular organelles known as lysosomes, where the liposomal membranes are thought to be degraded. From the lysosome, the liposomal lipid components migrate outward to become part of the cell's membranes and other liposomal components that resist lysosomal degradation (such as certain medications) may enter the cytoplasm.

Lipid exchange involves the transfer of individual lipid molecules from the liposome into the plasma membrane (and vice versa). With lipid exchange, the aqueous contents of the liposome do not enter the cell. For lipid exchange to take place, the liposomal lipid must have a particular chemistry in relation to the target cell. Once a liposomal lipid joins the cell membrane it can either remain in the membrane for a long time or be redistributed to a variety of intracellular membranes.

In very dilute solutions, lipid micelles may form instead of liposomes.

In the methods of the present invention, the cationic lipids of Formula I may further be conjugated to or mixed with or used in conjunction with a variety of useful molecules and substances such as proteins, peptides, growth factors and the like to enhance cell-targeting, uptake, internalization, nuclear targeting and expression. See, for example, U.S. Pat. Nos. 5,521,291, 5,547,932 and 5,693,509.

The method of the present invention can be applied to in vitro and in vivo transfection of eucaryotic cells or tissues including animal cells, human cells, insect cells, avian cells, fish cells, mammalian cells and the like. The method of this invention is useful in any therapeutic method requiring introducing of oligonucleotides into cells or tissues. In the method of the present invention, one or more antisense oligonucleotides are first contacted with one or more lipid formulations comprising an efficient amount of one or more cationic lipids of Formula I and optionally at least one neutral lipid to provide one or more antisense oligonucleotide-lipid aggregate complexes. For example, the contact can be made prior to the aggregate formation (from the cationic and neutral lipids) or subsequent to an initial lipid aggregate formation. In a preferred embodiment, the lipid aggregates of the cationic lipids and optional neutral lipids are formed first, then brought into contact with one or more antisense oligonucleotides. The antisense oligonucleotide will typically bind to the surface of the lipid aggregate as a result of the ionic attraction between the negatively charged antisense oligonucleotide and the positively charged surface of the lipid aggregate. Typically, the contact between the antisense oligonucleotide and the lipid aggregate that results in formation of a complex will be carried out at temperatures of from about 15° C. to about 45° C., preferably at room temperature. The length of time required to complete the formation of a complex will depend on the temperature as well as the nature of the antisense oligonucleotide and the lipid aggregate itself. When contact temperatures of about room temperature are used, the length of time to form a complex will be about 15 minutes to about 1 hour. Alternatively, the antisense oligonucleotide can be incorporated into the interior of liposomes prepared from the cationic lipids and optional neutral lipids of the invention by methods known to those of skill in the art. One method may involve encapsulation and can be carried out by a variety of techniques.

Following formation of antisense oligonucleotide-lipid aggregate complexes, the complexes are contacted with the cells to be transfected. Once adsorbed, the lipid aggregates, including the complexes, can either be endocytosed by a portion of cells, exchange lipids with the cell membranes or fuse with the cells as described above. Transfer or incorporation of the oligonucleotide part of the complex can take place via one of the above mentioned pathways. In particular, when a liposomal fusion takes place, the liposomal membrane and the antisense oligonucleotide-lipid aggregate complex combine with the intracellular fluid. Contact between the cells and the antisense oligonucleotide-lipid aggregate complexes, when carried out in vitro, will take place in a biologically compatible medium. The concentration of lipid can vary widely. Treatment of the cells with the antisense oligonucleotide-lipid aggregate complexes will generally be carried out at physiological temperatures (about 37° C.) for periods of time of from 1 to about 6 hours, preferably from 2 to 4 hours. For in vitro applications, the delivery of antisense oligonucleotides can be to any eucaryotic cell grown in culture. The cells are preferably mammalian cells, more preferably human cells.

Definitions

Useful alkyl groups include straight-chained and branched C₁₋₁₈ alkyl groups, preferably C₁₋₁₀ alkyl groups, more preferably C₁₋₅ alkyl groups. Typical C₁₋₁₈ alkyl groups include methyl, ethyl, propyl, isopropyl, butyl, sec-butyl, tert-butyl, 3-pentyl, hexyl, octyl, decyl, dodecyl, tetradecyl, hexadecyl and octadecyl groups.

Useful alkenyl groups are C₂₋₁₈ alkenyl groups, preferably C₂₋₁₀ alkenyl, more preferably C₂₋₆ alkenyl groups. Typical C₂₋₁₈ alkenyl groups include ethenyl, propenyl, isopropenyl, butenyl, sec-butenyl, hexenyl, octeneyl, decenyl, dodecenyl, tetradecenyl, especially 9-tetradecenyl, hexadecenyl, especially 9-hexadecenyl, and octadecenyl, especially 9-octadecenyl, groups.

Useful alkynyl groups are C₂₋₁₈ alkynyl groups, preferably C₂₋₁₀ alkynyl, more preferably C₂₋₆ alkynyl groups. Typical C₂₋₁₈ alkynyl groups include ethynyl, propynyl, butynyl, 2-butynyl, hexynyl, octynyl, decynyl, dodecynyl, tetradecynyl, hexadecynyl, and octadecynyl groups.

Typical heteroalkyl groups include any of the above-mentioned C₁₋₁₈ alkyl groups having one or more CH₂ groups replaced with 0 or S.

Typical heteroalkenyl groups include any of the above-mentioned C₂₋₁₈ alkenyl groups having one or more CH₂ groups replaced with O or S.

Typical heteroalkynyl groups include any of the above-mentioned C₂₋₁₈ alkynyl groups having one or more CH₂ groups replaced with O or S.

Typically alkylaminoalkyl groups are R₇—NH—R₈, wherein R₇ and R₈ are alkylene groups as defined above.

Useful aryl groups are C₆₋₁₄ aryl, especially C₆₋₁₀ aryl. Typical C₆₋₁₄ aryl groups include phenyl, naphthyl, phenanthryl, anthracyl, indenyl, azulenyl, biphenyl, biphenylenyl and fluorenyl groups.

Useful arylalkyl groups include any of the above-mentioned C₁₋₁₈ alkyl groups substituted by any of the above-mentioned C₆₋₁₄ aryl groups. Useful values include benzyl, phenethyl and naphthylmethyl.

Useful arylalkenyl groups include any of the above-mentioned C₂₋₁₈ alkenyl groups substituted by any of the above-mentioned C₆₋₁₄ aryl groups.

Useful arylalkynyl groups include any of the above-mentioned C₂₋₁₈ alkynyl groups substituted by any of the above-mentioned C₆₋₁₄ aryl groups. useful values include phenylethynyl and phenylpropynyl.

Useful halo or halogen groups include fluorine, chlorine, bromine and iodine.

Useful haloalkyl groups include C₁₋₁₀ alkyl groups substituted by one or more fluorine, chlorine, bromine or iodine atoms, e.g. fluoromethyl, difluoromethyl, trifluoromethyl, pentafluoroethyl, 1,1-difluoroethyl and trichloromethyl groups.

Useful hydroxyalkyl groups include C₁₋₁₀ alkyl groups substituted by hydroxy, e.g. hydroxymethyl, hydroxyethyl, hydroxypropyl and hydroxybutyl groups.

Useful alkoxy groups include oxygen substituted by one of the C₁₋₁₀ alkyl groups mentioned above.

Useful alkylthio groups include sulfur substituted by one of the C₁₋₁₀ alkyl groups mentioned above.

Useful acylamino groups are any acyl group, particularly C₂₋₆ alkanoyl or C₆₋₁₀ aryl(C₂₋₆)alkanoyl attached to an amino nitrogen, e.g. acetamido, propionamido, butanoylamido, pentanoylamido, hexanoylamido, and benzoyl.

Useful acyloxy groups are any C₁₋₆ acyl (alkanoyl) attached to an oxy (—O—) group, e.g. acetoxy, propionoyloxy, butanoyloxy, pentanoyloxy, hexanoyloxy and the like.

Useful alkylamino and dialkylamino groups are —NHR₉ and —NR₉R₁₀, wherein R₉ and R₁₀ are C₁₋₁₀ alkyl groups.

Aminocarbonyl group is —C(O)NH₂.

Useful alkylthiol groups include any of the above-mentioned C₁₋₁₀ alkyl groups substituted by a —SH group.

A carboxy group is —COOH.

A ureido group is —NH—C(O)—NH₂.

An amino group is —NH₂.

Optional substituents on R₁, R₂, R₃ and R₄ include any one of halogen, halo(C₁₋₆) alkyl, C₁₋₆ alkyl, C₂₋₆ alkenyl, C₂₋₆ alkynyl, hydroxy(C₁₋₆)alkyl, amino (C₁₋₆)alkyl, carboxy(C₁₋₆)alkyl, alkoxy(C₁₋₆)alkyl, nitro, amino, ureido, acylamino, hydroxy, thiol, acyloxy, alkoxy, carboxy, aminocarbonyl, and C₁₋₆ alkylthiol groups mentioned above. Preferred optional substituents include: hydroxy(C₁₋₆)alkyl, amino(C₁₋₆)alkyl, hydroxy, carboxy, nitro, C₁₋₆ alkyl, alkoxy, thiol and amino.

Pharmaceutically acceptable anion. Anions of inorganic or organic acids that provide non-toxic salts in pharmaceutical preparations.

Antisense Oligonucleotide. An antisense oligonucleotide is a DNA or RNA molecule or a derivative of a DNA or RNA molecule containing a nucleotide sequence which is complementary to that of a specific mRNA. An antisense oligonucleotide binds to the complementary sequence in a specific mRNA and inhibits or prevents translation of the mRNA. There are many known derivatives of such DNA and RNA molecules. See, for example, U.S. Pat. Nos. 6,031,086, 5,929,226, 5,886,165, 5,693,773, 6,054,439, 5,919,772, 5,985,558, 5,595,096, 5,916,807, 5,885,970, 5,877,309, 5,681,944, 5,602,240, 5,596,091, 5,506,212, 5,521,302, 5,541,307, 5,510,476, 5,514,787, 5,543,507, 5,512,438, 5,510,239, 5,514,577, 5,519,134, 5,554,746, 5,276,019, 5,286,717, 5,264,423, as well as WO96/35706, WO96/32474, WO96/29337 (thiono triester modified antisense oligodeoxynucleotide phosphorothioates), WO94/17093 (oligonucleotide alkylphosphonates and alkylphosphothioates), WO94/08004 (oligonucleotide phosphothioates, methyl phosphates, phosphoramiditates, dithioates, bridged phosphorothioates, bridge phosphoramidates, sulfones, sulfates, ketos, phosphate esters and phosphorobutylamines (van der Krol et al., Biotech. 6:958-976 (1988); Uhlmann et al., Chem. Rev. 90:542-585 (1990)), WO94/02499 (oligonucleotide alkylphosphonothioates and arylphosphonothioates), and WO92/20697 (3′-end capped oligonucleotides). Further, useful antisense oligonucleotides include derivatives such as S-oligonucleotides (phosphorothioate derivatives or S-oligos, see, Jack Cohen, Oligodeoxynucleotides, Antisense Inhibitors of Gene Expression, CRC Press (1989) which can be prepared, e.g., as described by Iyer et al. (J. Org. Chem. 55:4693-4698 (1990) and J. Am. Chem. Soc. 112:1253-1254 (1990)).

Complementary DNA (cDNA). A “complementary DNA,” or “cDNA” gene includes recombinant genes synthesized by reverse transcription of mRNA and from which intervening sequences (introns) have been removed.

Eucaryotic Cell. Eukaryotic cells can be of any type and from any source. Types of eukaryotic cells include epithelial, fibroblastic, neuronal, hematopoietic cells and the like from primary cells, tumor cells or immortalized cell lines. Sources of such cells include any animal such as human, canine, mouse, hamster, cat, bovine, porcine, monkey, ape, sheep, fish, insect, fungus and any plant including crop plants, ornamentals and trees.

Delivery is used to denote a process by which a desired compound is transferred to a target cell such that the desired compound is ultimately located inside the target cell or in, or on, the target cell membrane. In many uses of the compounds of the invention, the desired compound is not readily taken up by the target cell and delivery via lipid aggregates is a means for getting the desired compound into the cell. In certain uses, especially under in vivo conditions, delivery to a specific target cell type is preferable and can be facilitated by compounds of the invention.

Lipid Aggregate is a generic term which includes liposomes of all types both unilamellar and multilameller as well as micelles and more amorphous aggregates of cationic lipids mixed with neutral lipids.

Target Cell refers to any cell to which a desired compound is delivered, using a lipid aggregate as carrier for the desired compound.

Introducing is intended to include, e.g., transfecting, transforming, and delivering.

Transfection. Transfection is used herein to mean the delivery of an antisense oligonucleotide to a target cell, such that the antisense oligonucleotide is expressed or has a biological function in the cell. The term “expression” means any manifestation of the functional presence of the nucleic acid within the cell including, without limitation, both transient expression and stable expression. Functional aspects include inhibition of expression by oligonucleotides or protein delivery.

Kit refers to transfection or protein expression kits. Such kits are preferably used for introducing one or more oligonucleotides into one or more eucaryotic cells Such kits preferably comprise at least one compound selected from the group consisting of one or more cells, one or more antisense oligonucleotides, one or more lipid formulations of the invention, one or more buffering salts, one more culture media, one or more transfection enhancers, etc. Such kits may comprise a carrying means being compartmentalized to receive in close confinement one or more container means such as vials, test tubes and the like. Each of such container means comprises components or a mixture of components needed to perform transfection.

The invention will be further clarified by the following examples, which are intended to be purely exemplary of the invention. All reagents and media used in the examples were from Invitrogen Corporation, Life Technologies Division (Rockville, Md.) unless otherwise stated.

### EXAMPLES

Synthesis of Oligonucleotides

Synthesis and high-performance liquid chromatography (HPLC) purification of antisense phosphorothioate oligonucleotide (S—ODN) 5′-AACGTTGAGGGGCAT-3′ (SEQ ID NO: 1) complementary to the initiation codon of human c-myc mRNA and a scrambled phosphorothioate oligonucleotide containing the same base composition in random order 5′-GAACGGAGACGGTTT-3′ (SEQ ID NO:2) were performed as described by Wickstrom et al. (Proc. Natl. Acad. Sci. USA. 85:1028-1032 (1988) and Cancer Res. 52:6741-6745 (1992)).

Synthesis and high-performance liquid chromatography (HPLC) purification of antisense phosphorothioate oligonucleotide 5′-TCCCGCCTGTGACATGCATT-3′ (SEQ ID NO:3) complementary to the initiation codon of human c-raf, and a 7 bp mismatch phosphorothioate oligonucleotide 5′-TCCCGCGCACTTGATGCATT-3′ (SEQ ID NO:4) were performed as described by Monia et al. (Proc. Natl. Acad. Sci. U.S.A. 93:15481-15484 (1996)).

Cell Cultures

All cell lines were maintained at subconfluent levels and below passage 20 in a humidified incubator with a 5% CO₂ atmosphere at 37° C. for all experiments described. For transfection, cells were seeded onto 96-well microplates at specific plating densities (HeLa & HeLaS3: 2000 cells/well, HEK293: 3000 cells/well, CHOK1 & CHO-S: 1000 cells/well, K562: 1200 cells/well) in serum-containing medium. Adherent cells were seeded 24 hours before transfection and suspension cells were seeded 4 hours before transfection. Except for HeLa cells, all cells were then washed one time with serum-free growth medium and then treated for 4 hours in serum-free growth medium or with mixtures containing the tested transfection reagents and oligonucleotides. After 4 hours the appropriate growth medium containing 3× serum was added to the cells.

HeLa cells were grown in high-glucose Dulbecco's-modified Eagle's medium (DMEM: 4500 mg/L glucose, 862 mg/L L-alanyl-L-glutamine, 110 g/L sodium pyruvate) containing 10% (v/v) heat-inactivated, certified, fetal bovine serum (FBS).

Human endothelial kidney (HEK293) cells were plated in high-glucose Dulbecco's-modified Eagle's medium (DMEM) containing 10% (v/v) heat-inactivated, certified, fetal bovine serum (FBS), and 0.1 mM non-essential mino acids (NEAA).

Chinese Hamster Ovary (CHO-K1, adherent) and adapted for suspension growth (CHO-S) cells were grown in high-glucose DMEM, 10% FBS containing 0.1 mM NEAA, 1% proline, and 10% (v/v) heat-inactivated, certified, fetal bovine serum (FBS).

HeLaS3 (adapted for suspension growth) were grown in minimum essential medium with Earle's salts (S-MEM), 10% (v/v) heat-inactivated horse serum, and 4 mM L-glutamine.

K562 were grown in Iscove's modified Dulbecco's medium (IMDM: 4500 mg/L glucose, 862 mg/L L-alanyl-L-glutamine, 110 mg/L sodium pyruvate) containing 10% (v/v) heat-inactivated, certified, fetal bovine serum (FBS).

### Example 1

The cell lines HeLa, CHO-K1, CHO-S, 293F, K562, and HeLaS3 were transfected and assayed for a specific response to c-myc antisense oligonucleotides to investigate the potency of TR0 (a 1:2.5 w/w liposome formulation of the cationic lipid dimethyl dioctadecylammonium bromide (DDAB) and dioleyl phosphatidylethanolamine (DOPE)) as a non-toxic and specific means of delivery for antisense oligonucleotides. TR0 is sold under the trademark LIPOFECTACE™.

Transfection Procedure

The day before transfection, cells were plated in 96-well plates at an optimal seeding density according to each cell line described above. No antibiotics were used during these experiments. 200 nM of oligonucleotide (concentration calculated for a final volume of 100 μl) was added into 16 μl OPTI-MEM 1 Reduced Serum Medium. In a second tube, TR0 was diluted 1:5 in OPTI-MEM 1 Reduced Serum Medium and was incubated for 5-10 minutes at room temperature. Diluted TR0 was then added to diluted oligonucleotide (the final concentration of TR0 added per well was 8.4 μg/mL), mixed gently and incubated at room temperature for 15 minutes. 20 μl volumes of complexed TR0 and oligonucleotides were added to washed cells containing 80 μl of fresh serum-free medium. Complexes were incubated in serum-free medium for 4 hours at 37° C. 3× Serum-containing medium was then added to make a final concentration of 1× serum. 48 hours post-transfection, complexes were removed, cells washed and fresh growth media added. Cells were assayed for inhibition of proliferation at 24 hours, 48 hours, and 72 hours post-transfection. Both antisense and scrambled phosphorothioate oligonucleotides were transfected as described above. The control samples were prepared similarly without oligonucleotide or without oligonucleotide and TR0. The optimal concentration of TR0 was found to be between 5.6 μg/ml and 11.2 μg/ml.

Measurement of Cell Proliferation

Proliferation was measured with alamarBlue™ (Trek Diagnostics, Westlake, Ohio) which is a non-toxic redox indicator that yields a signal that can be detected with either fluorescent-based or absorbent based instrumentation in response to metabolic activity. alamarBlue™ was added to the cells at a 10% final volume of the reactions at 48 hours post-transfection. The absorbance of each well was read at two wavelengths, 570 nm and 600 nm, using a Molecular Devices Vmax® microplate reader and SOFTmax®Pro 3.1 software (Molecular Devices, Sunnyvale, Calif.). Plates were then placed in the CO₂ incubator and readings were taken at 24 hours, 48 hours, and 72 hours according to Voytik-Harbin et al. (J. Cell. Biochem. 67:478-491 (1997)). The percentage of inhibition of cellular proliferation was defined as the relative absorbance of sample versus untreated control cells.

Results

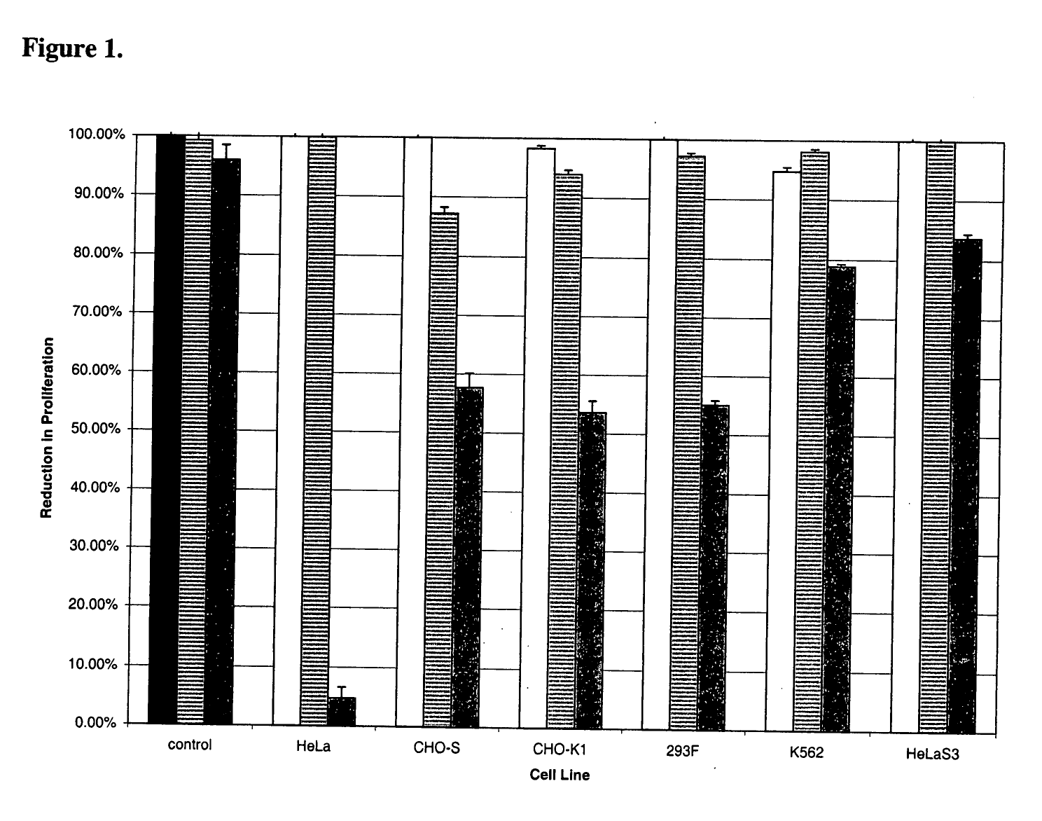

The results of the readings at 72 hours post-transfection are shown in FIG. 1. The numbers are presented according to the alamarBlue™ protocol. The results are expressed as a mean±SEM. Each assay represents the mean of replicates of 8 performed in a minimum of three separate experiments.

The results show that TR0-complexed ODN targeted to the c-myc start codon produces a significant reduction in cell growth and survival. In six different cell lines, TR0 consistently provided a specific inhibition of proliferation when compared to untreated cells. In HeLa cells, the inhibition was as great as 95% of the untreated sample. The variation in the magnitude of effect seen across cell lines can be understood as a function of the sensitivity of the specific cell line to c-myc down-regulation. Importantly, no cytotoxicity either with TR0 or with TR0 complexed to a scrambled ODN was observed with these complexes.

### Example 2

HeLa cell line was transfected and assayed for a specific response to c-myc antisense oligonucleotides using the following transfection reagents:

TR1 (LIPOFECTIN™): LIPOFECTIN™ (a 1:1 w/w liposome formulation of the cationic lipid N-[1-(2,3-dioleyloxy)propyl]-N,N,N-trimethylammonium chloride (DOTMA) and dioleyl phosphatidylethanolamine (DOPE in membrane filtered water) was diluted in OPTI-MEM 1 and incubated for 30 minutes at room temperature prior to complexation. Final concentration of LIPOFECTIN™ added was 0.3 μl/mL.

TR2 (CellFECTIN™): The final concentration of CellFECTIN™ (a 1:1.5 M/M liposome formulation of a cationic lipid tetramethylpalmitylspermine (TMTPS) and DOPE) added per well was 0.2 μg/mL.

TR3 (DMRIE-C™): The final concentration of DMRIE-C™ (a 1:1 M/M liposome formulation of a cationic lipid N-(2-hydroxyethyl)-N,N-dimethyl-2,3-bis(tetradecyloxy)-1-propanaminium bromide (DMRIE) and cholesterol) added per well was 0.15 μg/mL.

TR4 (LipofectAMINE™): The final concentration of LipofectAMINE™ (a 3:1 w/w liposome formulation of a polycationic lipid 2,3-dioleyloxy-N-[2-sperminecarboxamido)ethyl]-N,N-dimethyl-1-propanaminium (DOSPA) and DOPE) added per well was 0.3 μg/mL.

TR5 (LipofectAMINE 2000™): The final concentration of LipofectAMINE 2000™. added per cell was 0.2 μg/mL.

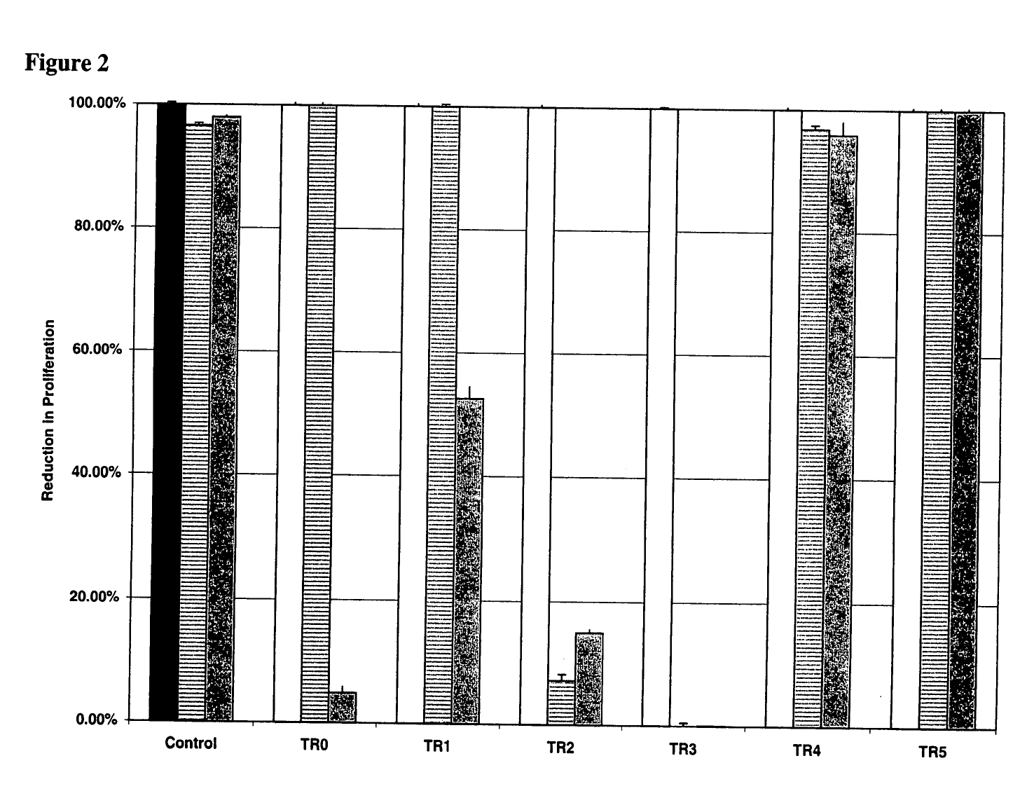

The transfections and measurement of cell proliferation followed the procedures described in Example 1. The results of the readings at 72 hours post-transfection are shown in FIG. 2. The numbers are presented according to the alamarBlue protocol. The results are expressed as a mean±SEM. Each assay represents the mean of replicates of 8 performed in a minimum of three separate experiments. The results for TR0 from Example 1 are presented in FIG. 2 for comparison.

FIG. 2 shows that TR0 produced the greatest reduction in cell growth and survival with little or no toxic effects. Of other five transfection reagents tested, only TR1 showed a specific inhibition of proliferation. However, TR1 only inhibited proliferation 40% to that of the untreated sample (a 95% inhibition seen with TR0). TR2 and TR3 showed an inhibition of proliferation in both the antisense/TR complex and the scrambled/TR complex. This effectively eliminates these reagents as viable for antisense research since a non-specific effect is not desirable. Complexes formed with TR4 and TR5 showed no response to antisense targeting.

### Example 3

### Western Blot Analysis

The ability of TR0/ODN complexes to inhibit c-Raf protein expression was examined by western blot analysis. Transfections were performed in 6-well plates using HeLa cells plated at 60,000 cells/well. Cells were treated for 6 hours with 200 nM of c-raf antisense or mismatch oligonucleotide complexed to TR0 (undiluted reagent was added for a final amount of 3 μl/well). The same treatment was repeated after 24 hours according to the procedure described by Lau et al. (Oncogene 16:1899-1902 (1998)). Supernatant was transferred to a fresh microfuge tube.

For immunoblot analysis, cells were harvested at 24 hours and 48 hours and washed with 1× PBS without Ca⁺⁺ or Mg⁺⁺. Cellular extracts were prepared using 1 mL of boiling lysis buffer (1% SDS, 1.0 mM sodium orthovanadate (Sigma-Aldrich, St. Louis, Mos.), and 10 mM Tris-HCl, pH 7.4). Typically, about 400 ng of protein were then separated and by electrophoresis on a 4-12% NuPage® Bis-Tris SDS-polyacrylamide mini-gel (Invitrogen Corporation, Carlsbad, Calif.). Once transferred to nitrocellulose, membranes were treated for 1 hour with a monoclonal antibody that specifically recognizes c-Raf kinase protein (BD Transduction Laboratories, Franklin Lakes, N.J.) at a dilution of 1:1,000. Detection was performed with WesternBreeze™ Kit (Invitrogen Corporation, Carlsbad, Calif.) and goat anti-mouse antibody (BD Transduction Laboratories, Franklin Lakes, N.J.). The control samples that received only TR0 without oligonucleotide were prepared accordingly.

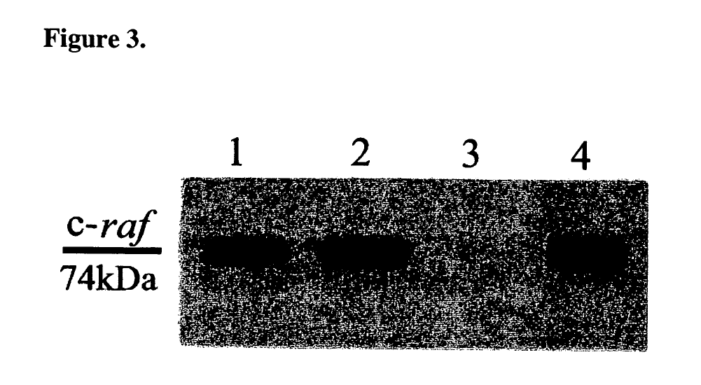

The results at 48 hours after treatment are shown in FIG. 3. Inhibition of c-Raf was observed only in the presence of the TR0/antisense c-raf complex. No inhibition of c-Raf expression was seen with the untreated samples, samples treated with TR0 alone, or with the TR0/mismatch ODN complex.

Those skilled in the art will recognize that while specific embodiments have been illustrated and described, various modifications and changes may be made without departing from the spirit and scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims. All publications, patent applications and patents cited herein are fully incorporated by reference.

What is claimed is:

1. A method for introducing one or more antisense oligonucleotides into one or more eucaryotic cells in vitro, comprising
(a) contacting said one or more antisense oligonucleotides with one or more lipid formulations comprising an effective amount of one or more cationic lipids of Formula I: 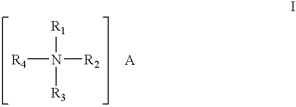
wherein
R₁ is a straight or a branched hydrocarbon chain of C₁₀₋₁₀₀ that is saturated or unsaturated;
R₂ is selected from the group consisting of a pair of electrons, hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted;
R₃ and R₄, independently of one another, are selected from the group consisting of hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted; wherein R₅ and R₆ are independently alkylene, alkenylene or alkynylene; and
A is an anion selected from Br⁻, Cl⁻, F⁻, I⁻, sulfate, nitrate, nitrite or a pharmaceutically acceptable anion when R₂ is not a pair of electrons;
and optionally at least one neutral lipid to form one or more antisense oligonucleotide-lipid aggregate complexes, and
(b) contacting said one or more cells with said one or more complexes.

2. The method according to claim 1, wherein when R₃ and R₄ are C₁₋₃ alkyl, and one of R₁ or R₂ is an unsaturated C₁₆₋₂₀ alkyl, the other one of R₁ and R₂ is not an unsaturated or saturated C₁₆₋₂₀ alkyl.

3. The method according to claim 1, wherein said one or more cells are not drug-resistant human breast carcinoma cells.

4. The method according to claim 1, wherein R₁ is a straight or branched hydrocarbon chain of C₁₀₋₃₀ that is saturated or unsaturated.

5. The method according to claim 4, wherein R₁ is a straight hydrocarbon chain of C₁₂₋₂₄ that is saturated or unsaturated; and R₂, R₃ and R₄ are independently selected from the group consisting of hydrogen, C₁₋₁₈ alkyl, C₂₋₁₈ alkenyl, C₂₋₁₈ alkynyl, C₄₋₁₈ heteroalkyl, C₄₋₁₈ heteroalkenyl, C₄₋₁₈ heteroalkynyl, C₆₋₁₂ aryl(C₁₋₁₈) alkyl and C₆₋₁₂ aryl, all of which can be optionally substituted.

6. The method according to claim 5, wherein R₁ is a straight hydrocarbon chain of C₁₄₋₂₀ that is saturated or unsaturated; R₂ is selected from the group consisting of hydrogen, C₆₋₁₈ alkyl, C₆₋₁₈ alkenyl, C₆₋₁₈ alkynyl, C₆₋₁₈ heteroalkyl, C₆₋₁₈ heteroalkenyl, C₆₋₁₈ heteroalkynyl, phenyl(C₆₋₁₈)alkyl, and phenyl; and R₃ and R₄ are independently selected from the group consisting of hydrogen, Cl -5 alkyl, C₂₋₆ alkenyl, C₂₋₆ alkynyl, C₂₋₅ heteroalkyl, C₂₋₅ heteroalkenyl, C₂₋₅ heteroalkynyl, phenyl(C₁₋₅)alkyl, and phenyl, all of which can be optionally substituted.

7. The method according to claim 6 wherein said cationic lipid of Formula I is dimethyldioctadecylammonium bromide (DDAB).

8. The method according to claim 6, wherein said cationic lipid of Formula I is dimethyldioctadecylammonium fluoride (DDAF).

9. The method according to claim 6, wherein said cationic lipid of Formula I is dimethyldioctadecylammonium chloride (DDAC).

10. The method according to claim 6, wherein said cationic lipid of Formula I is dimethyldioctadecylammonium iodide (DDAI).

11. The method according to claim 1, wherein said lipid formulation comprises a neutral lipid.

12. The method according to claim 1 1, wherein said neutral lipid is diacylphosphatidylethanolamine having 10-24 carbon atoms in the acyl group.

13. The method according to claim 12 wherein said neutral lipid is dioleylphosphatidylethanolamine (DOPE).

14. The method according to claim 1, wherein said cationic lipid is the cationic lipid of Formula II: 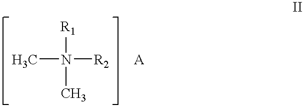
wherein
R₁ is a straight or a branched hydrocarbon chain of C₁₀₋₁₀₀ that is saturated or unsaturated;
R₂ is selected from the group consisting of a pair of electrons, hydrogen, alkyl, alkenyl, alkynyl, heteroalkyl, heteroalkenyl, heteroalkynyl, R₅—NHC(O)—R₆, R₅—C(O)—O—R₆, R₅—NH—C(O)—NH—R₆, R₅—NH—C(S)—NH—R₆, R₅—NH—C(NH)—NH—R₆, alkylaminoalkyl, arylalkyl, arylalkenyl, arylalkynyl, and aryl, all of which can be optionally substituted; wherein R₅ and R₆ are independently alkylene, alkenylene or alkynylene; and
A is an anion selected from Br⁻, Cl⁻, F⁻, F, sulfate, nitrate, nitrite or a pharmaceutically acceptable anion when R₂ is not a pair of electrons.

15. The method according to claim 14, wherein when one of R₁ or R₂ is an unsaturated C₁₆₋₂₀ alkyl, the other one is not an unsaturated or saturated C₁₆₋₂₀ alkyl.

16. The method according to claim 14, wherein R₁ is a straight or branched hydrocarbon chain of C₁₀₋₃₀ that is saturated or unsaturated.

17. The method according to claim 16, wherein R₁ is a straight hydrocarbon chain of C₁₂₋₂₄ that is saturated or unsaturated; and R₂ is selected from the group consisting of hydrogen, C₁₋₁₈ alkyl, C₂₋₁₈ alkenyl, C₂₋₁₈ alkynyl, C₄₋₁₈ heteroalkyl, C₄₋₁₈ heteroalkenyl, C₄₋₁₈ heteroalkynyl, C₆₋₁₂ aryl(C₁₋₁₈) alkyl and C₆₋₁₂ aryl, all of which can be optionally substituted.

18. The method of claim 17, wherein R₁ is a straight hydrocarbon chain of C₁₄₋₂₀ that is saturated or unsaturated; and R₂ is selected from the group consisting of hydrogen, C₆₋₁₈ alkyl, C₆₋₁₈ alkenyl, C₆—Is alkynyl, C₆₋₁₈ heteroalkyl, C₆₋₁₈ heteroalkenyl, C₆₋₁₈ heteroalkynyl, phenyl(C₆₋₁₈)alkyl, all of which can be optionally substituted.

19. The method according to claim 18, wherein R₁ is a straight hydrocarbon chain of C₁₄₋₂₀ that is saturated, and R₂ is selected from the group consisting of C₆₋₁₈ alkyl, C₆₋₁₈ heteroalkyl, C₆₋₁₈ heteroalkenyl, C₆₋₁₈ heteroalkynyl, and phenyl(C₆₋₁₈)alkyl, all of which can be optionally substituted.

20. The method according to claim 1, wherein A is selected from the group consisting of a halogen, a sulfate, a nitrate or a nitrite.

21. The method according to claim 20, wherein A is bromide.

22. The method according to claim 20, wherein A is fluoride.

23. The method according to claim 20, wherein A is chloride.

24. The method according to claim 20, wherein A is iodide.

25. The method according to claim 1, wherein said optional substituent is selected from the group consisting of halogen, halo(C₁₋₆) alkyl, C₁₋₆ alkyl, C₂₋₆ alkenyl, C₂₋₆ alkynyl, hydroxy(C₁₋₆)alkyl, amino(C₁₋₆)alkyl, carboxy(C₁₋₆)alkyl, alkoxy(C₁₋₆)alkyl, nitro, amino, ureido, acylamino, hydroxy, thiol, acyloxy, alkoxy, carboxy, aminocarbonyl, and C₁₋₆ alkylthiol.

26. The method according to claim 25, wherein said optional substituent is selected from the group consisting of hydroxy(C₁₋₆)alkyl, amino(₁₋₆)alkyl, hydroxy, carboxy, nitro, C₁₋₆ alkyl, alkoxy, thiol and amino.

27. The method according to claim 1, wherein said lipid formulation is present in an amount of about 0.1 μg/ml-5 mg/ml.

28. The method according to claim 27, wherein said lipid formulation is present in an amount of about 0.35-14 μg/ml.

29. The method according to claim 28, wherein said lipid formulation is present in an amount of about 2-13 μg/ml.

30. The method according to claim 29, wherein said lipid formulation is present in an amount of about 4.5-12 μg/ml.

31. The method according to claim 30, wherein said lipid formulation is present in an amount of about 5.6-11.2 μg/ml.

32. The method according to claim 1, wherein the antisense oligonucleotide is a deoxyribonucleic acid molecule.

33. The method according to claim 1, wherein the antisense oligonucleotide is a ribonucleic acid molecule.

34. The method according to claim 1, wherein the cells are selected from the group comprising HeLa, CHO-K1, CHO-S, 293F, K562, and HeLaS3.

35. A method for introducing one or more antisense oligonucleotides into one or more eucaryotic cells, comprising
(a) contacting said one or more antisense oligonucleotides with a lipid formulation comprising an effective amount of a cationic lipid and at least one neutral lipid to form one or more antisense oligonucleotide-lipid aggregate complexes, and
(b) contacting said one or more cells with said one or more complexes;
wherein the cationic lipid is selected from dimethyldioctadecylammonium fluoride (DDAF), dimethyldioctadecylammonium bromide (DDAB), dimethyldioctadecylammonium chloride (DDAC) or dimethyldioctadecylammonium iodide (DDAI).

36. The method according to claim 35, wherein the ratio of the cationic lipid and said neutral lipid is from about 1:5 to about 1:1.

37. The method according to claim 36, wherein said ratio is 1:2.5.

38. The method according to claim 35, wherein said neutral lipid is diacylphosphatidylethanolamine having 10-24 carbon atoms in the acyl group.

39. The method according to claim 38, wherein said neutral lipid is dioleylphosphatidylethanolamine (DOPE).

40. The method according to claim 39, wherein said lipid formulation is present in an amount of about 2-13 μg/ml.

41. The method according to claim 40, wherein said lipid formulation is present in an amount of about 4.5-12 μg/ml.

42. The method according to claim 41, wherein said lipid formulation is present in an amount of about 5.6-11.2 μg/ml.
